(19) United States
(12) Patent Application Publication
(10) Pub. No.: US 2021/0144945 A1
(43) Pub. Date: May 20, 2021

(54) Helianthus Plant Named 'BullHelidec 0201'

(71) Applicant: Hartwig Bull, Gonnebek (DE)

(72) Inventor: Hartwig Bull, Gonnebek (DE)

(21) Appl. No.: 17/096,493

(22) Filed: Nov. 12, 2020

Related U.S. Application Data
(60) Provisional application No. 62/974,135, filed on Nov. 15, 2019.

(51) Int. Cl. A01H 6/14; A01H 5/02; A01H 5/12

(52) U.S. Cl. CPC A01H 6/14; A01H 5/12; A01H 5/02

(57) ABSTRACT

A new and distinct variety of Helianthus plant having a compact growth habit and large, yellow colored blooms.

### CROSS REFERENCE TO RELATED APPLICATION(S)

This application claims the benefit of priority from U.S. Provisional Patent Application No. 62/974,135, filed Nov. 15, 2019, which is incorporated herein by reference in its entirety.

### BOTANICAL CLASSIFICATION

Helianthus decapetalus hybrid

### VARIETAL DENOMINATION

‘BullHelidec 0201’

### BACKGROUND OF THE INVENTION

The present invention comprises a new and distinct variety of Helianthus plant, botanically known as Helianthus decapetalus hybrid, and having the varietal denomination of ‘BullHelidec 0201’. The new variety was developed in August of 2017 in Gönnebek, Germany as a result of a mutation of Helianthus variety named ‘BullHeli 1216’ (unpatented). The purpose of the breeding program was to produce Helianthus varieties with improved growth and large flowers. The new variety was first asexually reproduced via in-vitro tissue culture cuttings in Gönnebek, Germany in February of 2018. ‘BullHelidec 0201’ is more compact, has more flowers, and an earlier flowering time than ‘BullHeli 1216’.

When compared to Helianthus variety named ‘Capenoch Star’ (unpatented), the varieties are similar in flower color, but ‘BullHelidec 0201’ is more compact, more self-branching, has a shorter crop time, smaller leaves, and larger flowers than ‘Capenoch Star’. Further, the following characteristics generally distinguish ‘BullHelidec 0201’ from other Helianthus varieties known to the breeder:
Compact growth habit;
Single, large flowers;
Small and attractive foliage;
Improved self-branching;
Desirable hardiness; and
More uniform crop growth.
   The new variety has been trial and field tested and has been found to retain its distinctive characteristics and remain true to type through successive propagations. The present invention has not been evaluated under all possible environmental conditions. The phenotype may vary with variations in environment without a change in the genotype of the plant.

### BRIEF DESCRIPTION OF THE DRAWINGS

The photographic drawings in the form of color photographs provided herein illustrate the new variety at approximately 17 weeks of age, with the colors being as nearly true as is possible with color illustrations of this type:

FIG. 1 shows an entire plant of the new variety; and

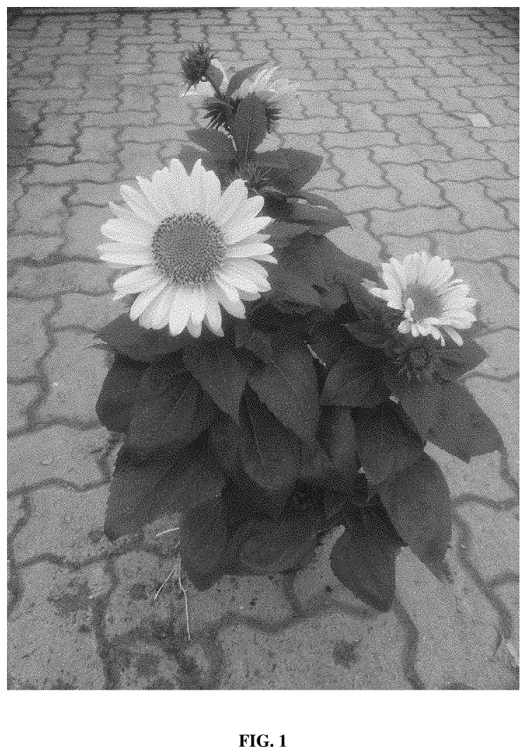

FIG. 2 shows a close-up view of a bloom of the new variety.

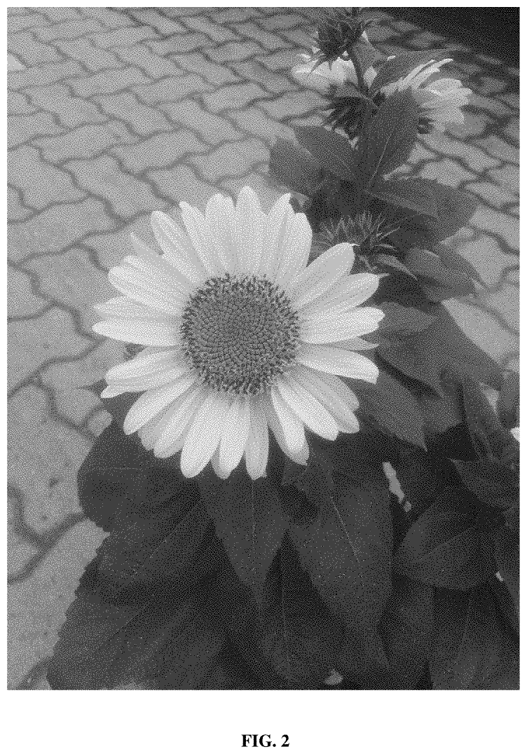

### DETAILED BOTANICAL DESCRIPTION

The following detailed description sets forth the characteristics of the new variety. The data which defines these characteristics was collected as a result of asexual reproductions via in-vitro tissue culture cuttings in Gönnebek, Germany The new variety was grown outside in 13.0 cm pots under normal field production conditions in Gönnebek, Germany. The color readings were taken outside under natural light and the variety was approximately 17 weeks of age when described. Color references are primarily to the 2015 R.H.S. Colour Chart of The Royal Horticultural Society of London, Sixth Edition, except where terms of ordinary significance are used.

### Plant

Time to initiate roots: About 14 days at about 18° C.
Time to develop roots: About 21-28 days at about 18° C.
Time to produce a finished flowering plant from a rooted cutting: About 8-12 weeks in a 13.0 cm container.
Rooting habit: Very freely branching with numerous roots of medium thickness.
Form: Upright.
Average height: Approximately 25.0-50.0 cm
Average width: Approximately 20.0-30.0 cm.
Disease/pest resistance: None observed to date.
Temperature tolerance: Tolerates temperatures from −15° C. to 35° C.
Drought tolerance: Average.
Branching habit: One dominate branch with a few side branches.
Lateral branches:

Number: 3-8 per plant.

Length: 20.0-30.0 cm.

Diameter: 3.0-12.0 mm.

Internode length: 2.0-10.0 cm.

Angle: Upright.

Strength: High.

Texture: Stable and rough.

Color: Yellow-Green Group RHS 144C, with older branches aging to Brown Group RHS 200C.

Foliage:

Arrangement: Alternate or opposite; single sessile.

Length 3.0-20.0 cm.

Width: 2.0-12.0 cm.

Shape of leaf: Aristate.

Apex: Acuminate.

Base: Obtuse.

Margin: Serrate.

Texture (both surfaces): Rough.

Color:

Young leaves:
Upper surface: Green Group RHS N138B.
Lower surface: Green Group RHS N138B.

Mature leaves:
Upper surface: Green Group RHS NN137B.
Lower surface: Green Group RHS 147B.

Petiole:

Length: 0.5-7.0 cm.

Diameter: Varies from from 0.2-1.0 cm throughout the length.

Texture: Rough and stable.

Color: Yellow-Green Group RHS 146C.

Veins:

Venation type: Palmate.

Color
Upper surface: Green Group RHS 139C.
Lower surface: Yellow-Green Group RHS 145A.

### Inflorescence

Flower arrangement: Composite; single.
Flower shape: Elliptic ray florets and tubular disc florets.
Natural flowering season: Plants freely flower from July-September in Gönnebek, Germany −7 to 10 weeks after planting.
Fragrance: Slightly sweet.
Average number: 5-20 per plant and 3-10 per branch.
Buds:

Length: 2.0-4.0 cm.

Diameter: 2.5-5.0 cm.

Color: Close to Green Group RHS 137B.
Inflorescence:

Diameter: 5.0-12.0 cm.

Height (depth): 2.0-5.0 cm.

Diameter of disc: 2.5-6.0 cm

Receptacle height: 1.0-2.0 cm.

Receptacle diameter: 3.0-6.0 cm.
Ray florets:

Number per inflorescence: Abundant—from 35-50.

Arrangement: In two or three whorls.

Length: 2.5-4.5 cm.

Width: 0.8-1.2 cm.

Shape: Elliptical, linear.

Apex: Rounded.

Base: Acute.

Margin: Entire.

Texture:
Upper surface: Smooth, soft.
Lower surface: Smooth, soft.

Color:

When opening:
Upper surface: Close to Yellow Group RHS 12A.
Lower surface: Close to Yellow Group RHS 12B.

At maturity:
Upper surface: Close to Yellow Group RHS 12A.
Lower surface: Close to Yellow Group RHS 12B.

Ground color description: Pure yellow.
Venation:

Appearance: Parallel.

Color:
Upper surface: Close to Yellow Group RHS 12A.
Lower surface: Close to Yellow Group RHS 12B.
Disc florets:

Number per inflorescence: Numerous—more than 100.

Arrangement: In the center of receptacle.

Length: 10.0-20.0 mm.

Width: 2.0 mm.

Shape: Tubular.

Apex: Five-pointed.

Color:
Immature: Yellow-light brown.
At maturity: Yellow with a shine of brown.
Phyllaries:

Number per inflorescence: Many—from 50-100

Arrangement: In 5 or 6 whorls.

Length: 1.0-2.0 cm.

Width: 4.0-5.0 mm.

Shape: Acicular.

Apex: Pointed, sharp.

Base: Fused.

Margin: Entire.

Texture:
Upper surface: Rough, uneven.
Lower surface: Rough, uneven.

Color:
Immature:
Upper surface: Close to Green Group 137D.
Lower surface: Close to Green Group 137B.

At maturity:
Upper surface: Close to Green Group 137D.
Lower surface: Close to Green Group 137B.

Reproductive organs:
Androecium:

Presence: On ray and disc florets.

Number (per floret): 2.

Filament length: 10.0 mm.

Filament color: Light yellow.

Anther:
Shape: Linear.
Length: 5.0 mm.
Color: Close to Brown Group RHS 200A.

Pollen:
Color: Yellow.
Amount: Plentiful.

Gynoecium:

Presence: Only in disc florets.

Pistil length: 12.0-15.0 mm.

Stigma:
Shape: Two-parted.
Color: Close to Yellow Group RHS 15A.

Style:
Length: 1.0 mm.
Color: Close to Yellow Group RHS 15A.

Seeds: None produced.

1. A new and distinct variety of Helianthus plant named ‘BullHelidec 0201’, as is herein described and illustrated.
